(19) United States
(12) Patent Application Publication
(10) Pub. No.: US 2010/0087000 A1
(43) Pub. Date: Apr. 8, 2010

(54) HEPATIC PROGENITOR CELL AND METHOD OF ISOLATING SAME

(75) Inventor: Randall Edwin McClelland, Chapel Hill, NC (US)

Correspondence Address: FOLEY AND LARDNER LLP;SUITE 500, 3000 K STREET NW, WASHINGTON, DC, 20007, US

(73) Assignee: Vesta Therapeutics, Inc.

(21) Appl. No.: 12/431,440

(22) Filed: Apr. 28, 2009

Related U.S. Application Data
(60) Provisional application No. 61/049,893, filed on May 2, 2008.

(51) Int. Cl. C12N 5/071

(52) U.S. Cl. 435/370; 435/402

(57) ABSTRACT

The isolation and proliferation of stem cells, particularly, hepatic stem cells continues to face technical hurdles. For example, colonies of stem cells, once isolated, tend to stop, or at least substantially slow, proliferating. The present invention provides a method to overcome this limitation, in part. The present invention also provides a method of providing and isolating a novel hepatic progenitor cell.

### CROSS REFERENCE TO RELATED APPLICATIONS

This application claims priority to U.S. Provisional Patent Application No. 61/049,893, filed May 2, 2008, the entire disclosure of which is incorporated herein by reference.

### FIELD OF THE INVENTION

The present invention relates generally to the isolation, expansion and/or differentiation of hepatic progenitor cells. More particularly, the present invention relates to a method of propagating and isolating hepatic progenitor cells.

### BACKGROUND OF THE INVENTION

The isolation and proliferation of stem cells, particularly, hepatic stem cells continues to face technical hurdles. For example, colonies of stem cells, once isolated, tend to stop, or at least substantially slow, proliferating. The present invention provides a method to overcome this limitation, in part. The present invention also provides a method of providing and isolating a novel hepatic progenitor cell.

### SUMMARY OF THE INVENTION

In one embodiment of the present invention, an isolated hepatic progenitor cell which does not express alpha-fetoprotein and does express EpCAM and albumin is provided, wherein the hepatic progenitor cell is adherent to the surface of a tissue culture plate, and wherein the level of albumin expression to EpCAM expression of said hepatic progenitor is about 0.7:1.0. The isolated surface-adherent hepatic progenitor cell is a precursor to a bipotent progenitor cell capable of giving rise to either hepatocytic cells or biliary cells and may further express CK19. The isolated hepatic progenitor cells have an average cell diameter ranging from about 12 μm to about 15 μm.

In another embodiment of the present invention, a method of providing a hepatic progenitor cell is provided, the method comprising: (a) plating a single cell suspension of hepatic cells on a planar surface and incubating the suspension to allow the hepatic cells to adhere to the planar surface; (b) propagating adherent cells in HK media to obtain one or more colonies of hepatic stem cells, each colony exhibiting a border; and (c) tilting the planar surface at an angle and allowing the cells of said one or more colonies to propagate beyond said border. The planar surface may be a tissue culture plate, optionally coated with one or more extracellular matrix proteins. The method can further comprise (d) isolating those cells which do not express alpha-fetoprotein and do express EpCAM and albumin, wherein the level of albumin expression to EpCAM expression of said hepatic progenitor ranges from about 0.5:1.0 to 0.9:1.0, preferably about 0.7:1.0.

Preferably, the colonies of hepatic stem cells have diameters between 30 μM and about 1200 μm, preferably between 100 μm and 700 μm, and most preferably between about 170 μm and 300 μm. The tilt angle can be periodically modified, and optionally modified mechanically by, for example, a rocker, shaker, or belly dancer. The tilt angle ranges between about 1 and about 90 degrees, preferably between about 15 and about 60 degrees, and most preferably between about 30 and about 60 degrees. The single cell suspension of hepatic cells is preferably plated in HK media further comprising serum. The border is relatively thick with cells comprising angioblasts, hepatoblasts, stellate cells, or a combination thereof.

In yet another embodiment of the present invention, a method of providing a colony of hepatic progenitor cells is provided, the method comprising: (a) obtaining a colony of hepatic stem cells on a planar surface, the hepatic stem cells having a negligible rate of proliferation; and (b) tilting the planar surface at an angle to allow the cells of the colony to resume proliferation. The method can further comprise (c) isolating those cells which do not express alpha-fetoprotein and do express EpCAM and albumin, wherein the level of albumin expression to EpCAM expression of said hepatic progenitor cell ranges from about 0.5:1.0 to 0.9:1.0.

Again, most preferably, the colonies of hepatic stem cells have a diameter between about 170 μm and 300 μm. The tilt angle can be periodically modified, and optionally modified mechanically by, for example, a rocker, shaker, or belly dancer. The tilt angle ranges between about 1 and about 90 degrees, preferably between about 15 and about 60 degrees, and most preferably between about 30 and about 60 degrees.

In further yet another embodiment of the present invention, a method of isolating a hepatic progenitor cell is provided, the method comprising: (a) plating a single cell suspension of hepatic cells on a tissue culture plate and incubating the suspension to allow the hepatic cells to adhere to the plate; (b) propagating the adherent cells in HK media to obtain colonies of hepatic stem cells; (c) physically disrupting the integrity of a colony of hepatic stem cells; (d) incubating the disrupted colonies to allow growth of cells therefrom; and (e) isolating those cells which do not express alpha-fetoprotein and do express EpCAM and albumin, wherein the level of albumin expression to EpCAM expression of said hepatic progenitor ranges from about 0.5:1.0 to 0.9:1.0.

### BRIEF DESCRIPTION OF THE DRAWINGS

FIG. 1 shows a 20× magnification of a colony of hepatic stem cells.

FIG. 2 shows a colony of hepatic stem cells (“original”) and cells that have proliferated therefrom upon tilting the original colony of hepatic stem cells for 10 days.

FIG. 3 shows a colony of hepatic stem cells prior to (3A) and after (3B) disruption. FIG. 3C shows cells that have proliferated upon disruption of the integrity of the original colony.

FIG. 4 shows bile caniculi (circled) before (4A) and after (4B) formation from mature hepatocytes. (40×)

### DETAILED DESCRIPTION OF THE PREFERRED EMBODIMENTS

The present invention provides a unique hepatic progenitor cell which is a precursor to a bipotent progenitor cell capable of giving rise to either hepatocytic cells or biliary cells. The invention also provides cellular compositions comprising the hepatic progenitor cell and methods for isolating the hepatic progenitor cell. In another embodiment, the present invention provides a method to obtain and propagate the unique hepatic progenitor cells. The method allows the manipulation of slow or “stalled” growth of colonies of hepatic stem cells in a way that the hepatic stem cells differentiate or de-differentiate and resume proliferation as the unique hepatic progenitors.

The term “hepatic progenitors,” as used herein, is broadly defined to include any cell that is not a fully mature (i.e., differentiated) hepatocyte or biliary cell. Hence, “hepatic progenitors” encompasses both hepatic stem cells and their progeny immature progeny. “Progeny” may include both self-replicating hepatic stem cells, hepatoblasts, bipotent progenitors therefrom, and progenitors committed to differentiate into a particular cell type (e.g., a committed biliary cell progenitor or hepatocyte).

Each of these aforementioned populations of hepatic cells (e.g., “stem cells,” “hepatoblasts,” or “hepatocytes”) can be identified by their respective size and/or specific arrangement of markers unique to the respective populations. See Table 1, below.

| | Hepatic Stem | | Adult Hepatocytes |
|---|---|---|---|
| | Cells | Hepatoblasts | (Adult Biliary Cells) |
| Size (μm) | 7-9 | 10-12 | 18-35 |
| | | | (n.t.) |
| EpCAM | +++ | ++ | −− |
| | | | (++) |
| AFP | −−− | +++ | −− |
| | | | (−−) |
| Albumin | + | ++ | +++ |
| | | | (−−) |
| CK19 | +++ | ++ | (++) |
| Claudin 3 | +++ | − | − |
| | | | (+) |
| Telomerase | +++ | +++ | ++ |
| | | | (n.t.) |
| Sonic and Indian | +++ | ++ | −− |
| Hedgehog | | | (−−) |
| I-CAM1 | −−− | +++ | ++ |
| | | | (+) |
| N-CAM | +++ | −−− | −− |
| | | | (−−) |
| MDR3 | − | − | +++ |
| | | | (+++) |
| P450-3A4 | −−− | −−− | +++ |
| | | | (−−) |

EpCAM = epithelial cell adhesion molecule; CK19 = cytokeratin 19, a biliary specific cytokeratin; I-CAM = intercellular adhesion molecule; NCAM = neuronal cell adhesion molecule; MDR3 = multidrug resistance gene isoform 3 (involved in bile transport) P450-C3A4 = cytochrome P450 3A4; Claudin 3 = tight junction protein (isoform 3), n.t = not tested.

Hepatic stem cells (HSCs) are pluripotent cells found in the ductal plates (also called limiting plates) in fetal and neonatal livers and in the Canals of Hering in pediatric and adult livers and showing evidence of self-replication with expression of telomerase and being capable of forming mature liver cells when transplanted. These cells are EpCAM+, NCAM+, ALB+, CK8/18+, CK19+, CD133/1+, and are negative for all hemopoietic markers tested (e.g., CD34, CD38, CD45, CD14) , mesenchymal cell markers (CD146, VEGFr, CD31) and for expression of P450s or alpha-fetoprotein. HSCs have a level of albumin expression to EpCAM expression of about 0.1:1.0. HSCs give rise to hepatoblasts and to committed (unipotent) biliary progenitors.

Hepatoblasts (HBs) are pluripotent cells found throughout the parenchyma of fetal and neonatal livers and as single cells or small aggregates of cells tethered to the ends of the Canals of Hering. HBs derive from the HSCs. HBs share many antigens present on HSCs but with important distinctions. For example, HBs do not express NCAM but rather ICAM1 and they express significant amounts of alpha-fetoprotein and fetal forms of P450s. These HBs give rise to the unipotent progenitors, the committed hepatocytic and biliary progenitors.

Hepatic Committed Progenitors are unipotent progenitors of either the hepatocytic and biliary lineages. Their antigenic profile overlaps with that of the HBs; however, biliary committed progenitors express CK19 but not AFP or ALB, whereas the hepatocytic committed progenitors express AFP and ALB but not CK19. Committed biliary progenitors derive directly from hepatic stem cells and also from hepatoblasts.

Mesenchymal Cells (MCs) include cells at various lineage stages of the many different mesenchymal cell types (listed as the mature cells and, in parentheses, their precursors): including stroma (mesenchymal stem cells), endothelia (angioblasts), stellate cells (stellate cell precursors), and various hemopoietic cells (hemopoietic stem cells)

While most, if not all, of the discussion and examples of hepatic progenitors herein will be with reference to human-derived cell populations (both adult and fetal), the teachings herein should not be limited to humans. In fact, one of ordinary skill in the art may be expected to apply the teachings herein to the expansion of hepatic progenitors from mammals, generally (e.g., mice, rats, dogs, etc.) Accordingly, the scope of the present invention is intended to include hepatic progenitors of any and all mammals.

It is also noted that hepatic progenitors suitable for in vitro isolation and propagation in accordance with the instant invention are not limited to those isolated or identified by any particular method. By way of example, methods for the isolation and identification of the hepatic progenitors have been described in, for example, U.S. Pat. No. 6,069,005 and U.S. patent application Ser. Nos. 09/487,318; 10/135,700; 10/387,547 and 11/560,049 the disclosures of which are incorporated herein in their entirety by reference.

Hepatic stem cells and hepatoblasts have characteristic antigenic profiles and can be isolated by protocols described previously. For example, hepatic stem cells and hepatoblasts share numerous antigens (e.g., cytokeratins 8, 18, and 19, albumin, CD133/1, and epithelial cell adhesion molecule (“EpCAM”) and are negative for hemopoietic markers (e.g., glycophorin A, CD34, CD38, CD45, CD14) and mesenchymal cell markers (e.g., CD146, CD31, VEGFr or KDR).

Alternatively, hepatic stem cells and hepatoblasts can be distinguished from each other by size (the stem cells are 7-9 μm; the hepatoblasts are 10-12 μm), by morphology in cultures (the stem cells form dense, morphologically uniform colonies, whereas the hepatoblasts form cord-like structures interspersed by clear channels, presumptive canaliculi), by distinctions in the pattern of expression of certain antigens (EpCAM is expressed throughout the hepatic stem cells but is confined to the cell surface in the hepatoblasts), or by distinct antigenic profiles (N-CAM is present in the hepatic stem cells, whereas alpha-fetoprotein (AFP) and ICAM1 are expressed by the hepatoblasts).

### Isolation of Hepatic Stem Cells

In one embodiment, primary hepatic stem cells are obtained from human livers in a manner described above. Briefly, a single cell suspension of whole liver hepatic cells is obtained and plated onto tissue culture plastic, alone, or on a matrix of extracellular proteins comprising collagen and laminin. The cells are then incubated in media comprising serum for a time necessary for the suspended cells to adhere to the plate (usually, 1-2 days).

Thereupon, the serum-containing media is removed and replaced with “Hiroshi Kubota's Media,” (HK), which is serum-free, and supplemented with specific growth factors. More specifically, HK is a serum-free basal medium (e.g., RPMI 1640) containing no copper, low calcium (<0.5 mM) and supplemented with insulin (5 μg/ml), transferrin/fe (5 μg/ml), high density lipoprotein (10 μg/ml), selenium (10-10 M), zinc (10-12 M) and 7.6 μE of a mixture of free fatty acids bound to purified albumin. The detailed methods for the preparation of this media have been published elsewhere, e.g., Kubota H, Reid L M, Proceedings of the National Academy of Sciences (USA) 2000; 97:12132-12137, the disclosure of which is incorporated herein in its entirety by reference.

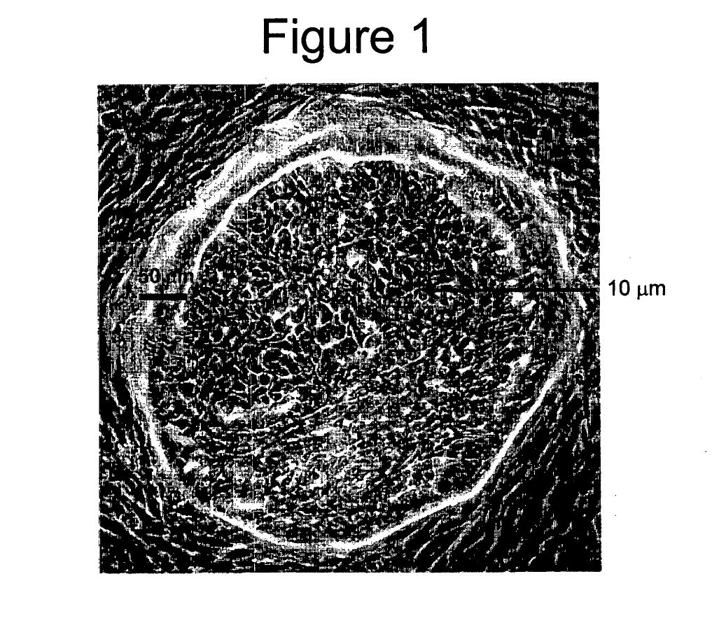

Under these conditions, over a period of 3-365 days, preferably 5-30 days, and more preferably 5-14 days, colonies of hepatic stem cells form relatively rapidly on the plate. As shown in FIG. 1, the colonies can be characterized morphologically as having a “thickened” “border” of cells encompassing a colony of “inner” cells. It has been found that the colony of “inner” cells have relatively weak adherence, if any, to the plate surface, but have relatively strong cell-cell contacts. The “inner” cells, however, do not float entirely off the plate, because they are collectively “tethered” to the place via the cells at the colony “border.”

The inner cells are mostly, if not entirely, comprised of hepatic stem cells. For example, these cells are less than 10 μm in diameter and are positive for EpCAM expression while negative for AFP expression. The cells at the border, however, are mostly non-hepatic cells, namely, angioblasts and stellate cells. However, some hepatic stem cells and hepatoblasts can be found at the “border” as well.

After about 14 days in culture, the colony of hepatic stem cells reach an average diameter of about 170 μm. Beneficially, this technique also works on colonies that have senesced, or at least, have negligible growth, for colonies between 100 μm-1200 μm in diameter. Negligible growth is defined here in as less than 0.5 cells divisions per day, preferable less than 0.25 cell division per day. As will be next discussed, the present invention provides a method to overcome the stalled proliferation.

### Isolation of Hepatic Progenitor Cells of the Invention

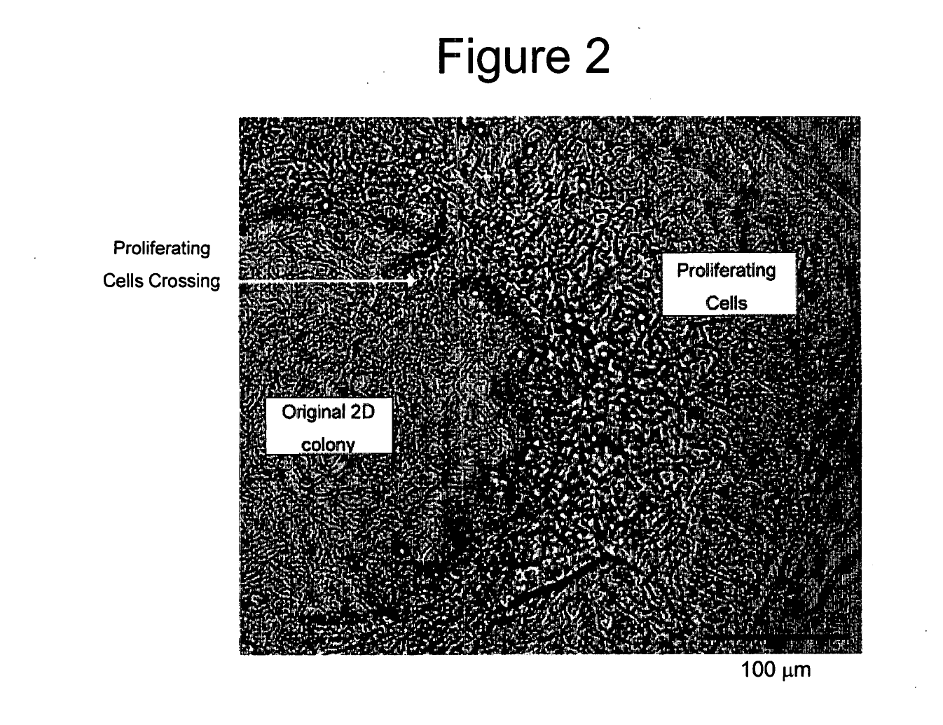

At or near the point and time the colony of hepatic stem cells reaches a rate of negligible growth, the present inventors have learned that tilting the planar surface (i.e., the plate) to which the colony is attached leads to resumption to proliferation (FIG. 2). Typically, proliferation resumes between 2 hours-10 days, preferably 8 hours-5 days, and more preferably 1-3 days after tilting. The planar surface may be tilted at an angle of about 1 to about 90 degrees, preferably from about 15 to about 60 degrees, and most preferably from about 30 to about 60 degrees. Table 2 (below) shows the relative degree of cell proliferation as a function of tilt angle.

Range of Tilt (degrees)

| | | |
|---|---|---|
| 0-15 | 16-30 | 31-90 |

Cell Proliferation

| | | |
|---|---|---|
| Low | medium | High |

In addition to a “permanent” tilt angle, the present invention may also be practiced by periodically modifying the tilt either manually or mechanically. Mechanical devices for periodic tilt adjustment include “rockers,” “shakers,” and “belly dancers.”

As an alternative to tilting, the present invention also provides a method of obtaining the unique progenitor cells by physically disrupting the integrity of a colony of hepatic stem cells. For example, once a hepatic stem cell colony is fully, or near fully, formed, the integrity of the outer edge, or border, of cells may be disrupted by use of a an object (e.g., a knife) to break-up the cohesive structure of a colony.

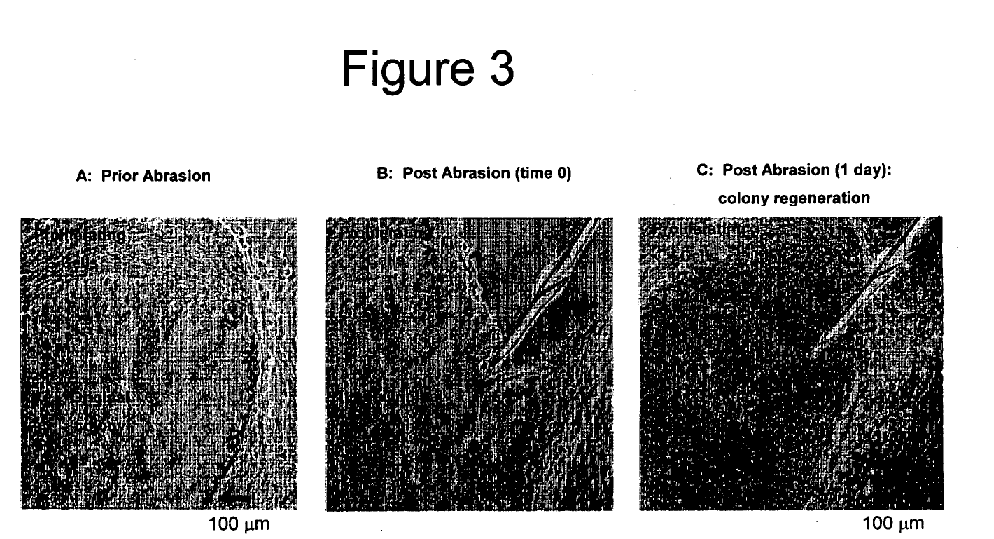

FIGS. 3A and B show photographs of a colony of hepatic stem cells before and after disruption, respectively. The colony of cells shown in FIG. 3A has ceased growing. Upon disruption of the integrity of the colony, however, the unique progenitor cells of the present invention are shown occupying the vacant “slice” in the upper right corner of FIG. 3B (FIG. 3C) after only one day post-disruption.

Without being held to or bound by theory, it is presently believed that the hepatic stem cells, when tethered within the confines of the “border,” are either physically limited in their ability to further proliferate and/or are subject to anti-proliferative signals from the colony. Hence, by tilting the plate, it is believed that the hepatic stem cells escape the structural and/or chemical limitations of the original colony of cells, and are able to resume proliferation. This hypothesis is also believed to explain why greater cell proliferation is observed when the plate is tilted to a greater degree. Table 2.

The cells that appear post-tilting (i.e., beyond the original colony border) appear to share characteristics of both hepatic stem cells and hepatoblasts. For example, the cells are EpCAM-positive and AFP-negative like hepatic stem cells. However, while these cells and hepatic stem cells are both albumin-positive, the post-tilt cells have noticeably greater albumin, about 2-5 times more. Compared with EpCAM, for example, the post-tilt cells have a ratio of between about 0.5:1.0 to 0.9:1.0 EpCAM:albumin, whereas hepatic stem cells have a ratio of 0.1:1.0 EpCAM:albumin. As well, the post-tilt cells have an average diameter of 12-15 μm (similar to hepatoblasts) and adhere to the plate surface, whereas, as mentioned above, the hepatic stem cells are not adherent per se, but are “tethered” to the plate via cells along the border of the colony.

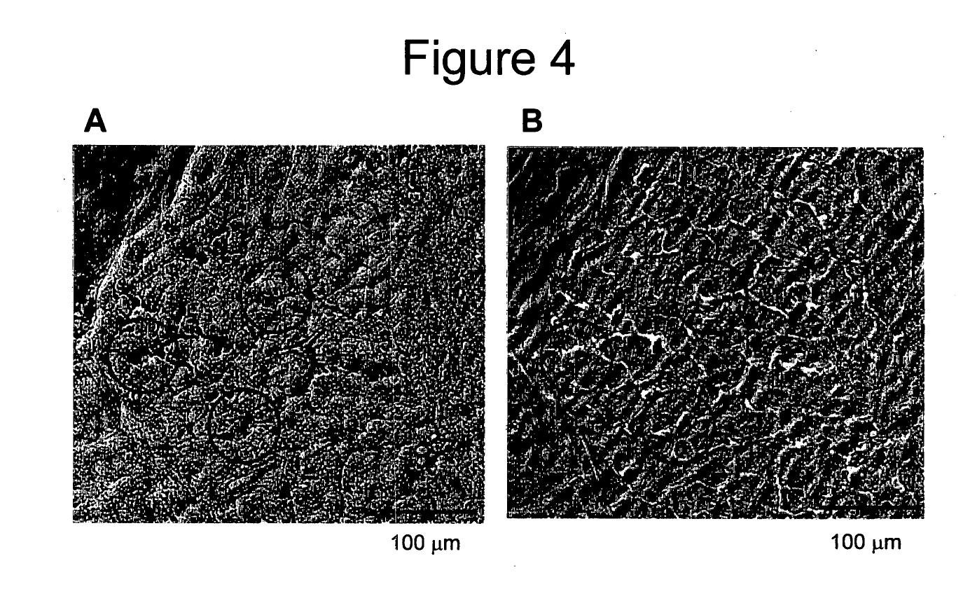

In either case, the post-tilt cells have the potential to differentiate into mature hepatocytes. Indeed, after about 7 days in culture, bile caniculi (FIG. 4B) appear between the hepatocytes (FIG. 4A) that have expanded beyond the original culture border. As only mature hepatocytes are capable of forming these structures, the appearance of bile caniculi is evidence that the progenitor cells that appear post-tilting are, in fact, progenitor cells. As well, because the progenitor cells are AFP-negative and EpCAM positive, the novel progenitor cells isolated by the inventive method are either (i) a pluripotent “stem cell” or (ii) a bipotent progenitor cell arising from a hepatic stem cell, but not yet a hepatoblast, which are AFP-positive.

While the invention has been described in connection with specific embodiments thereof, it will be understood that it is capable of further modifications and this application is intended to cover any variations, uses, or alterations of the invention following. In general, the principles of the invention and including such departures from the present disclosure as come within known or customary practice within the art to which the invention pertains and as may be applied to the essential features hereinbefore set forth and as follows in the scope of the appended claims.

What is claimed is:

1. An isolated hepatic progenitor cell which does not express alpha-fetoprotein and does express EpCAM and albumin, wherein the level of albumin expression to EpCAM expression of said hepatic progenitor ranges from about 0.5:1:0 to about 0.9:1.0.

2. The isolated hepatic progenitor cell of claim 1 which is a precursor to a bipotent progenitor cell capable of giving rise to either hepatocytic cells or biliary cells.

3. The isolated hepatic progenitor of claim 1 which further expresses CK19.

4. The isolated hepatic progenitor cell of claim 1 which has an average cell diameter ranging from about 12 μm to about 15 μm.

5. A method of providing a hepatic progenitor cell, comprising:
(a) plating a single cell suspension of hepatic cells on a planar surface and incubating the suspension to allow the hepatic cells to adhere to the planar surface;
(b) propagating adherent cells in HK media to obtain one or more colonies of hepatic stem cells, each colony exhibiting a border; and
(c) tilting the planar surface at an angle and allowing the cells of said one or more colonies to propagate beyond said border.

6. The method of claim 5 in which the planar surface is a tissue culture plate.

7. The method of claim 6 in which the tissue culture plate is coated with one or more extracellular matrix proteins.

8. The method of claim 5 further comprising (d) isolating those cells which do not express alpha-fetoprotein and do express EpCAM and albumin, wherein the level of albumin expression to EpCAM expression of said hepatic progenitor is 0.7:1.0.

9. The method of claim 5 in which the colonies of hepatic stem cells have a diameter greater than about 170 μm.

10. The method of claim 5 in which the tilt angle is periodically modified.

11. The method of claim 7 in which the tilt angle is periodically modified mechanically.

12. The method of claim 8 in which the tilt angle is periodically modified mechanically with a rocker, shaker, or belly dancer.

13. The method of claim 5 in which the tilt angle ranges between about 1 and about 90 degrees.

14. The method of claim 13 in which the tilt angle ranges between about 15 and about 60 degrees.

15. The method of claim 14 in which the tilt angle ranges between about 30 and about 60 degrees.

16. The method of claim 5 in which the single cell suspension of hepatic cells is plated in HK media further comprising serum.

17. The method of claim 5 in which the border comprises angioblasts, hepatoblasts, stellate cells, or a combination thereof

18. A method of providing a colony of hepatic progenitor cells comprising:
(a) obtaining a colony of hepatic stem cells on a planar surface, the hepatic stem cells having a negligible rate of proliferation; and
(b) tilting the planar surface at an angle to allow the cells of the colony to resume proliferation.

19. The method of claim 18 further comprising (c) isolating those cells which do not express alpha-fetoprotein and do express EpCAM and albumin, wherein the level of albumin expression to EpCAM expression of said hepatic progenitor is about 0.7:1.0.

20. The method of claim 18 in which the planar surface is a tissue culture plate.

21. The method of claim 20 in which the tissue culture plate is coated with one or more extracellular matrix proteins.

22. The method of claim 18 in which the colonies of hepatic stem cells have a diameter greater than about 170 μm.

23. The method of claim 18 in which the tilt angle ranges between about 1 and about 60 degrees.

24. The method of claim 23 in which the tilt angle ranges between about 30 and about 60 degrees.

25. A method of isolating a hepatic progenitor cell, comprising:
(a) plating a single cell suspension of hepatic cells on a tissue culture plate and incubating the suspension to allow the hepatic cells to adhere to the plate;
(b) propagating the adherent cells in HK media to obtain colonies of hepatic stem cells;
(c) physically disrupting the integrity of a colony of hepatic stem cells;
(d) incubating the disrupted colonies to allow growth of cells therefrom; and
(e) isolating those cells which do not express alpha-fetoprotein and do express EpCAM and albumin, wherein the hepatic progenitor cell is adherent, and wherein the level of albumin expression is substantially greater than the albumin expressed in a hepatic stem cell.

26. An isolated hepatic progenitor cell which does not express alpha-fetoprotein and does express EpCAM and albumin, wherein the hepatic progenitor cell is adherent to the surface of a tissue culture plate, and wherein the level of albumin expression to EpCAM expression of said hepatic progenitor ranges from about 0.5:1:0 to about 0.9:1.0.
